(19) United States
(12) Patent Application Publication
(10) Pub. No.: US 2012/0227750 A1
(43) Pub. Date: Sep. 13, 2012

(54) DENTAL IMPLANT FOR TREATMENT OF SLEEP APNEA

(75) Inventor: John H. Tucker, Fairview, PA (US)

(73) Assignee: ADVANCED AIRWAY DESIGN LLC, Aspinwall, PA (US)

(21) Appl. No.: 13/510,771

(22) PCT Filed: Nov. 19, 2010
(86) PCT No.: PCT/US10/57335

§ 371 Date: May 18, 2012

Related U.S. Application Data
(60) Provisional application No. 61/263,123, filed on Nov. 20, 2009.

(51) Int. Cl. A61F 5/56

(52) U.S. Cl. 128/848

(57) ABSTRACT

An apparatus for use in treatment of sleep apnea includes: a first body for use on an upper jaw; and a second body which is matingly engaged by the first body wherein the second body is used on a lower jaw. The first body has a first member and the second body has a second member. The member slidably engages the second member and the second body has a wall which slides along a wall of the first body which moves the second body. The second body is secured to at least one dental implant on the lower jaw.

A method of treating sleep apnea, includes: providing an apparatus having a first body for use on an upper jaw; and a second body which is matingly engaged by the first body wherein the second body is used on a lower jaw. The first body has a first member and the second body has a second member. The first member slidably engages the first member with the second member. The second body is mounted onto the lower jaw by securing the second body onto at least one dental implant in the lower jaw. A wall of the first body is slid along a wall of the second body to move the second body.

### CLAIM OF PRIORITY

This application claims priority from U.S. Provisional Patent Application Ser. No. 61/263,123, filed on Nov. 20, 2009, which is hereby incorporated by reference.

### BACKGROUND OF THE DISCLOSURE

The present disclosure generally relates to apparatus and methods for treating sleep apnea and/or related breathing disorders, and more specifically relates to an apparatus for placement in the oropharyngeal region of a human and methods using such apparatus for treating sleep apnea and/or snoring.

Sleep apnea is a sleep-related breathing disorder that is thought to affect between 1-10% of the adult population. Recent epidemiologic data indicate that 2% of women and 4% of men between the ages of 30 and 60 years meet the minimum diagnostic criteria for sleep apnea syndrome, representing more than 10 million individuals in the United States. It is a disorder with significant morbidity and mortality, contributing to increased risk of hypertension, cardiac arrhythmias, stroke, and cardiovascular death. Another common sleep-related breathing disorder is snoring, which may be associated with or independent of sleep apnea.

The present disclosure has been developed to aid in the treatment of hypopnea and apnea that occur due to pathological disturbances in the sleep process. One of the main reasons of the sleep disturbance is the relaxation of the tongue and pharyngeal walls to varying degrees during the several stages of sleep. When fully awake, these tissues have normal tone as air passes in and out of the lungs during respiration. However, during sleep, the musculature supporting these tissues relaxes. As air is inspired, the tongue and posterior walls of the pharynx collapse, causing snoring or more seriously, causing partial or complete obstruction of the airway.

Obstructive sleep apnea occurs due to a collapse of soft tissue within the upper airway during sleep. The ongoing force of inspiration serves to generate increasingly negative pressure within the pharynx, causing further collapse. The lack of respiration results in inadequate blood oxygenation, and rising carbon dioxide levels. The cardiovascular response produces an increase in the blood pressure and pulse. Cardiac arrhythmias often occur. The carbon dioxide increase and oxygen desaturation triggers a transition to a lighter sleep stage, usually without wakefulness. This transition brings a return to tonicity of the muscles of the upper airway, allowing normal breathing to resume. The person then returns to deeper stages of sleep and the process is repeated. The disease is quantified in terms of respiratory disturbances per hour. Mild disease begins at ten per hour, and it is not uncommon to find patients with indices ranging from sixty events to over one hundred or more per hour.

Not surprisingly, sleep is extremely fragmented and of poor quality in persons suffering from sleep apnea. As a result, such persons typically feel tired upon wakening and may fall asleep at inappropriate times during the day. All aspects of quality of life, from physical and emotional health, to social functioning are impaired by obstructive sleep apnea.

Several methods for treating snoring of a patient have been developed, which include embedding an implant into a soft palate. These methods reduce the audibility of snoring but do not address the more serious condition of sleep apnea.

Furthermore, these conventional devices and treatments continue to suffer poor cure rates. The failures lie in their inability to maintain patency in the retropalatal region and retroglossal region (the caudal margin of the soft palate to the base of the epiglottis). The poor success rates combined with high morbidity, contribute to an ongoing need for more effective treatments for sleep apnea and/or snoring.

Also, treatments such as CPAP are uncomfortable and therefore are poorly tolerated in some instances, and do not always provide successful results. Accordingly, the present disclosure provides a dental apparatus and method effective to treat, to substantially eliminate or at least reduce the occurrence of, sleep apnea and/or snoring.

The present disclosure also provides a dental appliance for treatment of sleep apnea which is secured to a dental implant.

### SUMMARY OF THE DISCLOSURE

The present disclosure provides a variety of dental appliances sized and structured to attach to a variety of dental implants or a dental implant mesiostructure and/or bar and placed in an oropharyngeal region of a human. The dental apparatus' function is to reduce or minimize the occurrence of sleep apnea and/or snoring.

A patient will have an apparatus in accordance with the present disclosure located within the patient's oropharyngeal region in order to treat, control, reduce or even substantially eliminate the occurrence of sleep apnea and/or snoring.

Snoring and sleep apnea are often caused by a combination of narrowness and low muscle tone of the upper airways. The tongue falls back and may obstruct the airway, possibly leading to an arousal reaction and disturbing the normal sleeping pattern. Other portions of the oropharyngeal region may also collapse. For example, the lateral walls of the oropharyngeal region often become excessively lax and block a free flow of air during respiration. When the patient is supine, for example, and the patient is asleep lying on his/her back, the relaxed tongue moves inferior (downward) and posterior (backward), and/or the lateral walls of the oropharyngeal region collapse inwardly resulting in a narrower pharynx relative to when the patient is upright. One cause for the narrowing of the pharynx in the supine position could be that the oropharyngeal region and the hypopharyngeal region, which have low consistencies, collapse because of lack of direct hard tissue support.

The apparatus of the present disclosure is preferably designed to provide direct support to at least some of these tissues when the patient is supine and asleep. For example, the apparatus of the present disclosure is structured so that when placed in the given position in oropharyngeal region, the apparatus will advance the lower jaw or mandible forward, and/or push the lateral walls away from one another thereby holding the airway patent or open during the time the human is naturally sleeping.

In all embodiments of the disclosure, the apparatus is structured to closely and flexibly conform to the size and contours of at least a portion of the oropharyngeal region.

The apparatus is preferably structured to maintain a radial force or pressure, for example, a substantially constant radial force or pressure, against the lower jaw or mandible advancing it anteriorly. The pressure of the apparatus maintained against the lower jaw or mandible is sufficient to maintain patency of the oropharyngeal region during natural sleep in a supine position, and preferably exerts pressure less than that exerted by the surfaces of the oropharyngeal region during swallowing.

The apparatus is designed and structured to allow substantially normal functioning of the oropharyngeal and pharyngeal regions, while maintaining the structural integrity of the apparatus over a long period of time. An important consideration in the design of the present apparatus includes the requirement that the apparatus substantially maintain its structural integrity and strength despite the highly dynamic, peristaltic motion of the oropharyngeal and hypopharyngeal regions.

The appropriate amount of force necessary to provide adequate support to maintain patency and consistent air flow through the oropharyngeal region during sleep, while allowing for the dynamic motion of the pharyngeal region and normal function of the oropharyngeal region and/or hypopharyngeal region, may vary between patients. Therefore, preferably the apparatus is available in a range of radial forces and sizes in order to suit different individuals.

In accordance with one aspect of the disclosure, an apparatus for use in treatment of sleep apnea includes: a first body for use on an upper jaw; and a second body which is matingly engaged by the first body wherein the second body is used on a lower jaw. The first body includes a first member and the second body includes a second member. The first member slidably engages the second member and the second body has a wall which slides along a wall of the first body which moves the second body. The second body is secured to at least one dental implant on the lower jaw.

In accordance with another aspect of the disclosure, a method of treating sleep apnea includes: providing an apparatus having a first body for use on an upper jaw; and a second body which is matingly engaged by said first body wherein the second body is used on a lower jaw. The first body includes a first member and the second body includes a second member. The first member slidably engages the first member with the second member. The method further includes mounting the second body onto the lower jaw by securing the second body onto at least one dental implant in the lower jaw; and sliding a wall of the first body along a wall of the second body to move the second body.

In accordance with yet another aspect of the disclosure, a dental appliance assembly for use in treatment of sleep apnea has an upper appliance which is mounted on an upper jaw; a lower appliance which is mounted on a lower jaw; wherein at least one of the upper appliance and the lower appliance is secured to at least one dental implant formed in one of the upper and lower jaws.

Another aspect of the disclosure is the apparatus includes an appliance sized and structured to be placed in a given position in the oropharyngeal region, and to be effective, when placed in the given position, to treat sleep apnea and/or snoring in a patient or user.

Another aspect of the disclosure is the appliance of the present invention is advantageously structured to have an enhanced ability to provide support against collapse of the oropharyngeal region during natural sleep, as well as to allow proper closure of an airway in the oropharyngeal region during swallowing.

Another aspect of the disclosure is the apparatus is designed in such a manner as to substantially prevent interfering with the normal functioning of the tissue around the apparatus.

Still another aspect of the disclosure is an appliance which moves the lower jaws anteriorly, and splints the airway and keeps it open and prevents collapse of the airway.

Yet another aspect of the disclosure is that the appliance is secured and attached to dental implants or a dental implant mesiostructure and/or bar within the user's jaws.

These and other aspects of the present disclosure will become apparent after a reading and understanding of the following detailed description.

### BRIEF DESCRIPTION OF THE DRAWINGS

FIG. 1 is a perspective view of an upper and lower appliance attached to upper and lower jaw mock-ups;

FIG. 2 is a front elevational view of the upper and lower appliance of FIG. 1;

FIG. 3 is a side elevational view showing a screw and pin arrangement for moving the lower appliance relative to the upper appliance;

FIG. 4 is a side elevational view showing the upper appliance being lowered onto the lower appliance;

FIG. 5 is a perspective view showing the upper appliance being removed from the lower appliance;

FIG. 6 is a side elevational view showing the lower appliance moved forward (to the left of FIG. 6);

FIG. 7 is a top plan view of the underside of the lower appliance;

FIG. 8 is a top plan view of the lower appliance showing metal pins;

FIG. 9 is a front elevational view of the lower appliance;

FIG. 10 is a front elevational of the lower jaw mock-up with dental implants;

FIG. 11 shows a front elevational view of the lower appliance being lowered onto dental implants in a lower jaw mock-up; and

FIG. 12 is a top plan view showing the upper and lower appliances.

### DETAILED DESCRIPTION OF THE DISCLOSURE

Snoring is the sound of partially obstructed breathing during sleep. While snoring can be harmless, it can also be the sign of a more serious medical condition known as Obstructive Sleep Apnea (OSA). When Obstructive Sleep Apnea occurs, the tongue and soft palate collapse onto the back of the throat and completely block the airway, which restricts the flow of oxygen. The condition known as Upper Airway Resistance Syndrome (UARS), is midway between primary snoring and true obstructive sleep apnea. People with UARS suffer many of the symptoms of OSA but require special sleep testing techniques.

Appliance therapy is used for patients with primary snoring or mild OSA who do not respond to, or are not appropriate candidates for treatment with behavioral measures such as weight loss or sleep-position change. Oral appliances can also be used with patients who refuse treatment, or are not candidates for tonsillectomy and adenoidectomy, cranofacial operations, or tracheostomy.

Oral appliances that treat snoring and obstructive sleep apnea are small plastic/acrylic, custom manufactured and fitted devices that are worn on upper and lower jaws in the mouth, similar to orthodontic retainers or sports mouth guards. These appliances are classified as a class II Medical Device, cleared by the FDA in order to treat sleep apnea. Their function is to help prevent the collapse of the tongue and soft tissues in the back of the throat, keeping the airway open during sleep and promoting adequate air intake. Oral appliances may be used alone or in combination with other means of treating OSA, including general health and weight management, surgery, or CPAP. One such example of an oral appliance is shown in U.S. Pat. No. 6,877,513 to Scarberry, et al.; which is hereby incorporated by reference.

Oral appliance therapy involves the selection, fitting, and use of a specially designed oral appliance worn during sleep that maintains airway patency for the user. Oral appliances can be classified by mode of action or design variation.

Tongue retaining appliances function by holding the tongue in a forward position by means of a suction bulb. When the tongue is in a forward position, it serves to keep the back of the tongue from collapsing during sleep and obstructing the airway in the throat.

Oral appliance therapy functions by maintaining the lower jaw (mandible) in a protruded/forward position during sleep. This serves to open the airway by indirectly pulling the tongue and connected soft tissues forward that also stimulates activity and increases muscle tonality in the oral cavity. As it holds the lower jaw and other structures in a stable position this prevents the airway from obstructing and causing the patient or user to have an apnea event. The present disclosure is described in relation to the oral appliances, but it can be used with other appliances, such as the tongue retaining appliances described above.

Oral appliance therapy has several advantages over other forms of therapy: oral appliances are comfortable and easy to wear—most people find that it only takes a couple of weeks to become acclimated to wearing the appliance; oral appliances are small and convenient making them easy to carry when traveling; treatment with oral appliances is reversible and non-invasive.

The present disclosure pertains to an apparatus for enhancing the patency of a patient's upper airway, and, in particular, an apparatus for stabilizing anatomical features associated with the upper airway so as to minimize airway obstruction or restriction.

The anatomic feature positioning includes lower jaw or mandible positioning and stabilization using upper and lower appliances. It should be noted that the benefits of the oral appliance of the present disclosure, i.e., to enable reduced pressure levels for the pressure support therapy, are applicable not only to patients suffering from OSA, hypopnea, UARS, and other conditions associated with upper airway instability, but also to any patient receiving a pressure support therapy for any reason.

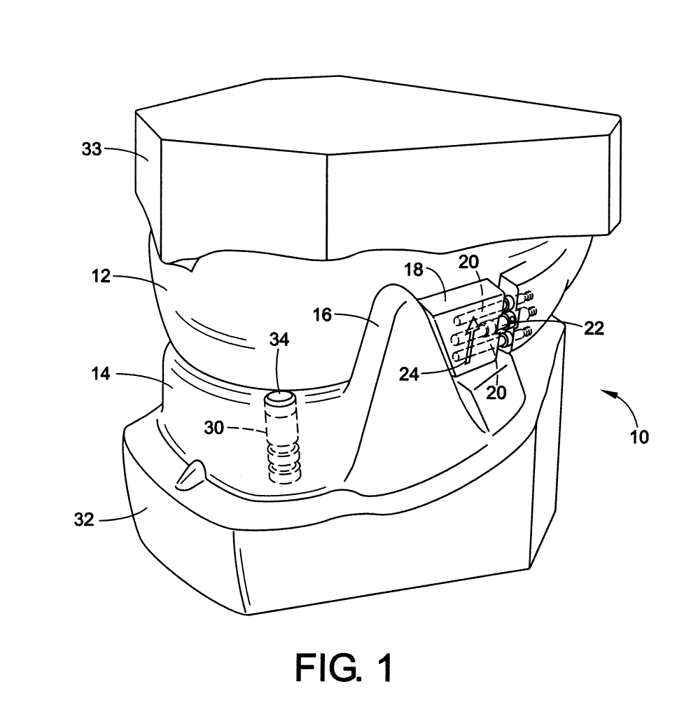

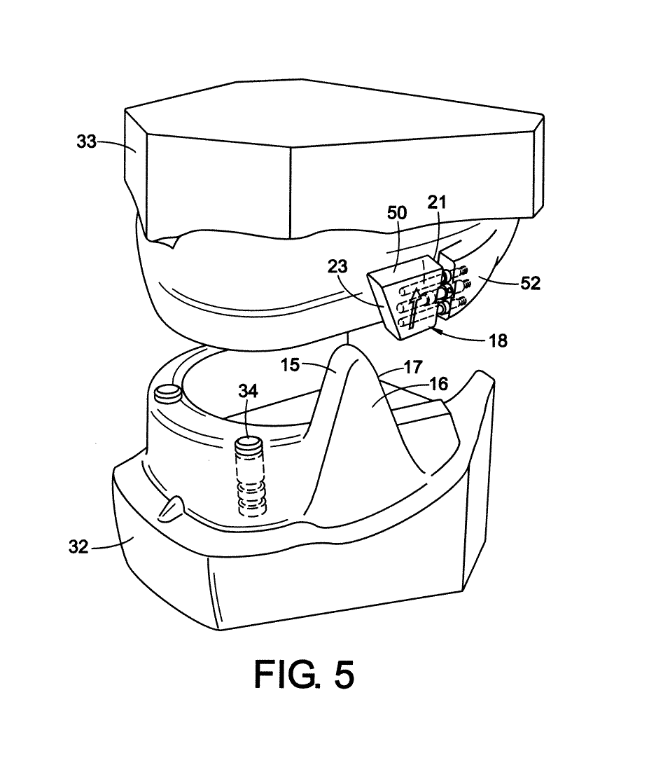

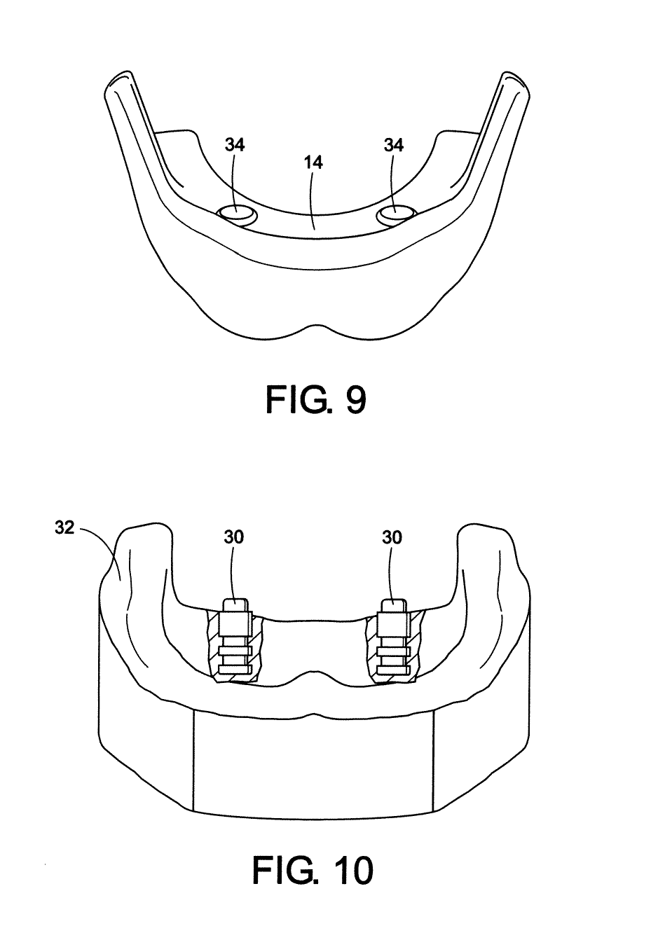

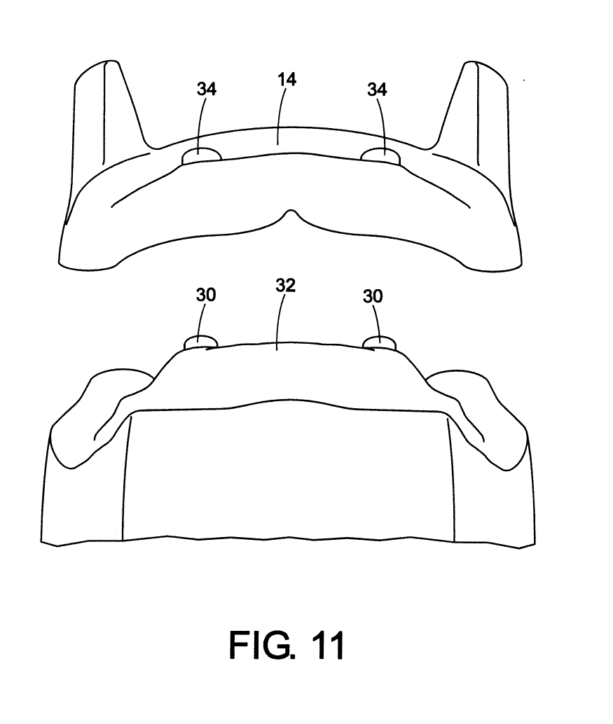

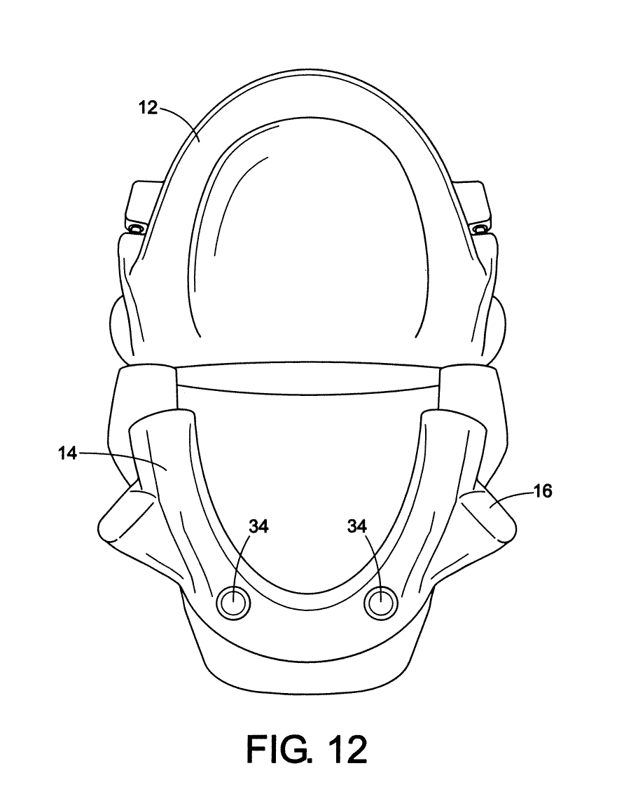

FIGS. 1-12 illustrate an exemplary embodiment of an intraoral appliance 10 according to one embodiment of the present disclosure. The appliance 10 shown in the Figures is shown for illustration purposes with respect to use with a dental implant. The disclosure is not limited to the appliances shown in FIGS. 1-12; rather, any dental appliance for treating sleep apnea is contemplated for use with dental implants.

Referring to FIG. 1, appliance 10 includes a formed unitary, generally rigid upper appliance body 12 and a mating lower appliance body 14, which may be molded from such suitable biocompatible materials as polyurethane or polycarbonate plastic, or other suitable materials without departing from the scope of the disclosure. Each of the upper and lower bodies 12, 14 are conformed to be mounted or placed on the upper and lower jaws of the user.

Intraoral appliance 10 is received within an oral cavity of a user and is retained therein by interfitting with the user's lower jaw (shown as lower jaw mock-up 32 in FIG. 1) and upper jaw (shown as upper jaw mock-up 33). Specifically the upper and lower bodies 12, 14 are received by the upper and lower jaws 33, 32 of a user, respectively, much as dentures would be. Accordingly, respective lower and upper appliances 12, 14 are shaped to conform generally to a user's lower and upper jaws. In an exemplary embodiment, each appliance body 12, 14 can be formed by a quantity of impression forming material, such as ethylene vinyl acetate (EVA) or any other suitable material. Impression material is used to form exact impressions of the user's jaws (including but not limited to Dentate, Partially Dentate and fully Edentulas). Upon cooling or setting, the EVA retains the dentition impression, thereby creating a custom fitted appliance.

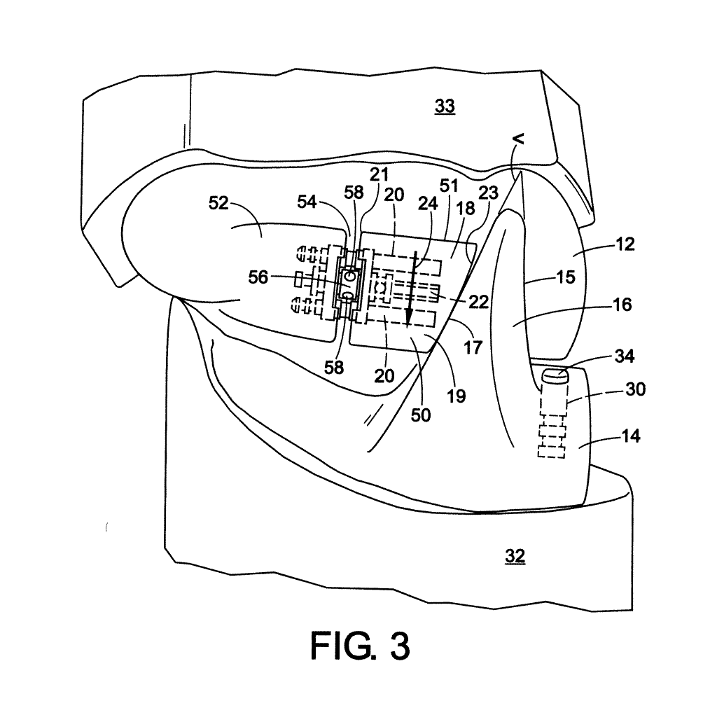

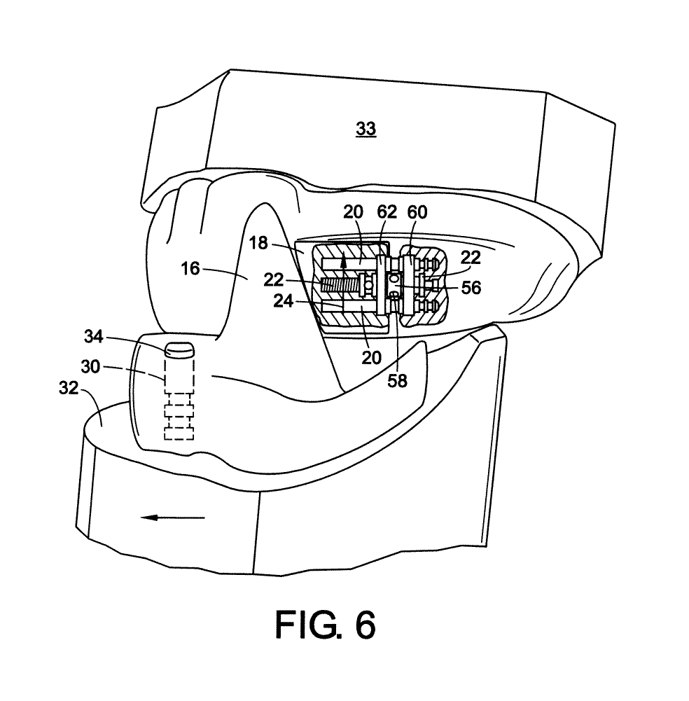

In a preferred embodiment of the present disclosure, lower jaw or appliance body 14 is provided in a fixed position relative to upper appliance or jaw body 12. Furthermore, referring to FIGS. 3-6, lower appliance body 14 is offset in an anterior or forward direction (toward the right in FIG. 3 and toward the left in FIG. 6), relative to the position of upper body 12, so that when the user's upper and lower jaws 33, 32 are engaged with the upper and lower appliance bodies, respectively, the lower appliance 14 and corresponding structures, including the jaw or mandible, are disposed forward (i.e. to the left of FIG. 6) of their neutral position with respect to the upper jaw and upper appliance 12.

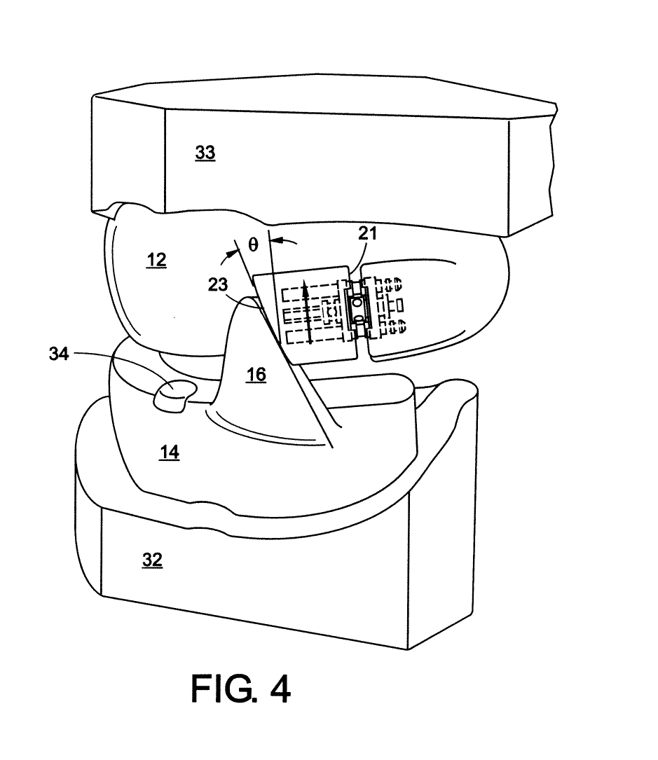

Referring now to FIG. 3, the lower body 14 can have an angled or fin shaped protrusion 16 which matingly or slidably engages an angled protrusion or wall 18 formed on the upper body 12. Specifically, fin-shaped protrusion 16 has a first generally straight wall 15 and a second angled wall 17 formed at an angle with respect to each other. For example, wall 17 is disposed at an angle _{<}of about 45 degrees with respect to wall 15. However, the angle can range from about 30 degrees to 60 degrees or so without departing from the scope of the disclosure. The fin shaped protrusion 16 keeps the lower jaw or mandible in a forward or extended position with respect to the upper jaw or mandible. Likewise, protrusion 18 has a body 19 with a generally straight wall 21 and an angled wall 23 on opposite sides of the protrusion. Angled wall 23 is also positioned at about a 45 degree angle θ (FIG. 4) with respect to generally straight wall 21. However, the angle may also vary from about 30 degrees to about 60 degrees.

Protrusion 18 has an extending member 50 which includes a series or pair of parallel pins 20 which are housed within wall 51 of member 50. The pins also extend into wall 52 of body 12. Wall 52 is spaced from wall 51. The pins are used to align or index and locate the extending member 50. A screw 22 is centrally positioned between the pair of pins 20 to facilitate movement of the protrusion 18. Screw 22 extends into wall 52 as well. Plates 60, 62 are positioned with walls 51, 52 and have openings through which the pins and screws extend. A recess or gap 54 is formed between walls 51 and 52 and have openings to provide access to pins 20 and screw 22. A housing 56 is provided which is mounted to the body of screw 22 and has a series of spaced apart holes 58 therein to facilitate access to and rotation of the screw. Housing 56 has a diameter greater than an outer diameter of the screw. A pin or rod (not shown) can be inserted into one of the holes, and then the housing is rotated in the direction of arrow 24 to move wall 51 away from wall 52. In turn, angled wall 23 pushes against angled wall 17. Lower appliance 14 then moves forward; i.e., to the right of FIG. 3 in a lateral direction. Screw 22 is also turned and rotated in the direction of arrow 24 (downward) formed on wall 51 to move the wall 51 and lower jaw forward (to the right of FIG. 3). Fin 16 has angled wall 17 which slides along angled wall 23, which pushes the appliance body 14 forward. Once the desired setting is obtained, the screw maintains the position of the lower appliance body 14 with respect to the upper appliance body 12. The dentist can adjust the lower jaw for the patient, or the patient can perform the adjustment himself or herself. This mechanism can be used with any suitable oral appliance used in the treatment of obstructive sleep apnea.

Although a rotary screw configuration is shown in FIGS. 3-6 for moving the upper and lower body members relative to one another, it is to be understood that other mechanisms for adjusting the position of the lower body member relative to the upper body member are also contemplated by the present disclosure, which result in offsetting the lower jaw with respect to the upper jaw to treat sleep apnea.

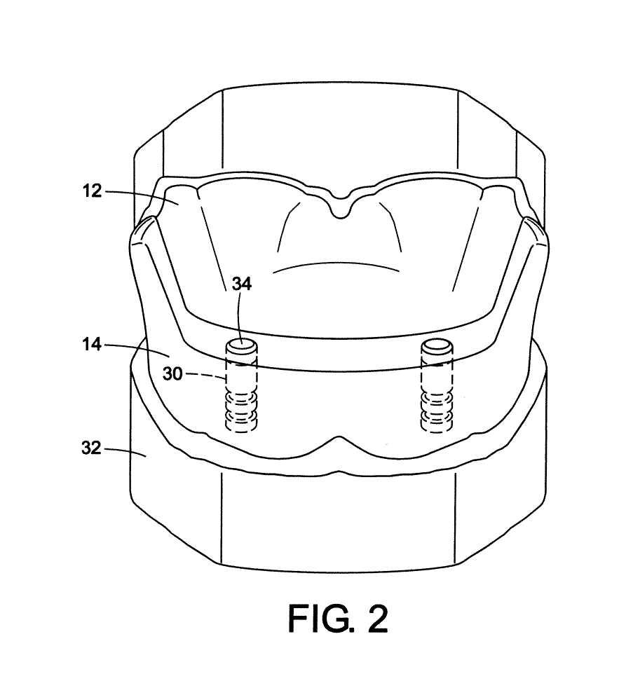

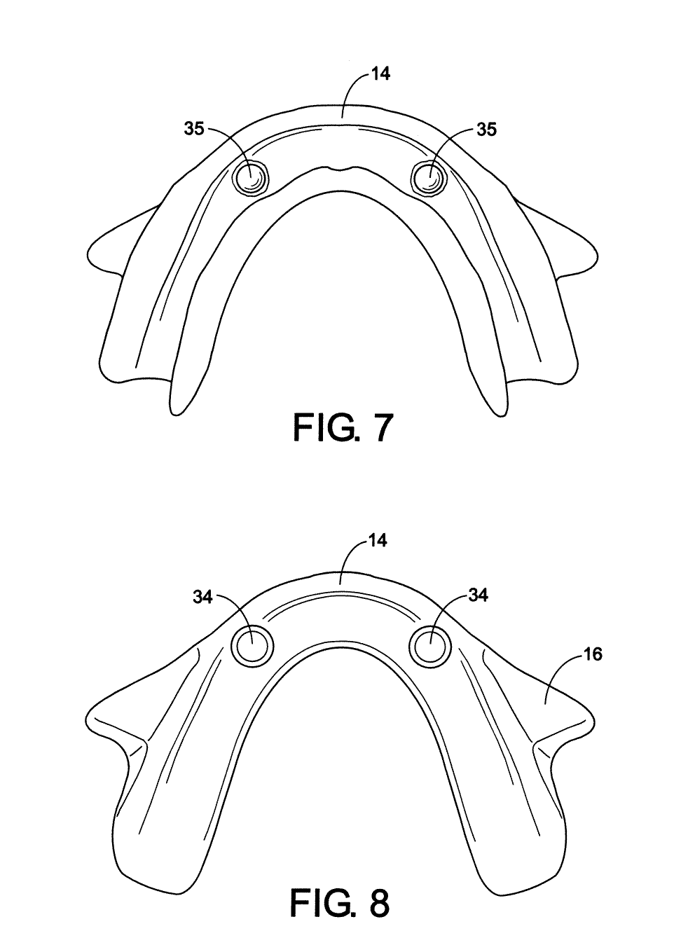

Referring specifically now to FIGS. 7-12, dental implants 30 are shown as in the lower jaw mock-up 32. Any dental implant can be used with the present disclosure; such as pins or rods or bars. That is, the dental implants are used to secure the appliance to either the upper jaw or lower jaw. For example, metal or silver colored pins 34 formed on the lower body 14 are secured, attached or snapped onto the implants 30 as shown in FIGS. 2, 8 and 9. The underside 35 of the pins 34 which fit onto the upper surface of the implants 30 are shown in FIG. 7. The upper and lower body appliances are secured to the implants 30 much as dentures would be secured to upper and lower jaws. The implants 30 can be brass pins which project from the jaws and receive the silver pins from the appliances. However, any type of attachment can be used to attach the appliance to the implants, such as two-way bars. Also, any type of implant can be used. Also, any number of implants can be used, such as one or two or more implants.

The exemplary embodiment has been described with reference to the present disclosure. Obviously, modifications and alterations will occur to others upon reading and understanding the preceding detailed description. It is intended that the exemplary embodiment be construed as including all such modifications and alterations insofar as they come within the scope of the above description.

1. An apparatus for use in treatment of sleep apnea, comprising:
a first body for use on an upper jaw;
a second body which is matingly engaged by said first body wherein said second body is used on a lower jaw;
wherein said first body comprises a first member and said second body comprises a second member;
wherein said first member slidably engages said second member and said second body has a wall which slides along a wall of said first body which moves said second body; and
said second body is secured to at least one dental implant on said lower jaw.

2. The apparatus of claim 1, wherein said first member of said first body comprises a first wall and a second wall formed at an angle with respect to said first wall.

3. The apparatus of claim 2, wherein said second member of said second body comprises a first wall and a second wall formed at an angle with respect to said first wall.

4. The apparatus of claim 1, wherein said first member comprises at least one alignment pin extending through said first member.

5. The apparatus of claim 4, further comprising a screw extending between said first member and a third member of said first body.

6. The apparatus of claim 5, wherein when said screw is rotated, said first member moves with respect to said third member.

7. The apparatus of claim 6, wherein said first member is moved into contact with said second member of said second body and moves said second body in a lateral direction by pushing on said second member.

8. The apparatus of claim 1, wherein said second body comprises a pin which is received by said at least one dental implant.

9. The apparatus of claim 8, wherein said second body pin has an underside surface which is secured to a top surface of said at least one dental implant.

10. A method of treating sleep apnea, comprising:
providing an apparatus having a first body for use on an upper jaw; and a second body which is matingly engaged by said first body wherein said second body is used on a lower jaw; wherein said first body comprises a first member and said second body comprises a second member; wherein said first member slidably engages said first member with said second member;
mounting said second body onto said lower jaw by securing said second body onto at least one dental implant in said lower jaw; and
sliding a wall of said first body along a wall of said second body to move the second body.

11. The method of claim 10, further comprising providing at least one alignment pin extending through said first member.

12. The method of claim 11, comprising providing a third member spaced apart from said first member on said first body.

13. The method of claim 12, further comprising providing a screw extending between said first member and a third member of the first body.

14. The method of claim 13, comprising rotating said screw to move said first member into contact with said second member to move said second member and the lower jaw in a lateral direction.

15. The method of claim 10, wherein mounting of said second body onto said lower jaw comprises securing a pin on said second body onto said at least one dental implant in said lower jaw.

16. A dental appliance assembly for use in treatment of sleep apnea; comprising:
an upper appliance which is mounted on an upper jaw;
a lower appliance which is mounted on a lower jaw;
wherein at least one of said upper appliance and said lower appliance is secured to at least one dental implant formed in one of said upper and lower jaws.

17. The appliance assembly of claim 16, wherein said at least one of said upper and said lower appliance comprises a pin which is secured to said at least one dental implant.
